(19) United States
(12) Patent Application Publication
(10) Pub. No.: US 2024/0017084 A1
(43) Pub. Date: Jan. 18, 2024

(54) fNIRS for Dosing Transcranial Magnetic Brain Stimulation

(71) Applicant: The Florida State University Research Foundation, Inc., Tallahassee, FL (US)

(72) Inventors: Frank Andrew Kozel, Tallahassee, FL (US); Kevin Johnson, Tallahassee, FL (US)

(21) Appl. No.: 18/222,108

(22) Filed: Jul. 14, 2023

Related U.S. Application Data
(60) Provisional application No. 63/389,422, filed on Jul. 15, 2022.

(51) Int. Cl. A61N 2/00; A61N 2/02

(52) U.S. Cl. CPC A61N 2/006; A61N 2/02

(57) ABSTRACT

A system may measure, via a functional Near-Infrared Spectroscopy (fNIRS) device, a first set of measurements of a prefrontal cortex region of a patient. The system may perform, via a transcranial magnetic stimulation (TMS) device, TMS treatment to the prefrontal cortex region for therapeutic effect. The fNIRS device may measure a second set of measurements of the prefrontal cortex region. The system includes a processor to determine an applied dosing of the TMS treatment based on the first set of measurements and/or the second set of measurements. The system may output control feedback to the TMS device or visualization to a display to adjust one or more parameters or localization of the TMS treatment based on the applied dosing. The system personalizes the treatment dosing of TMS more accurately over the prefrontal cortex by calibrating the dose to the prefrontal cortex based on measurements from the fNIRS device.

### CROSS-REFERENCE TO RELATED APPLICATIONS

This application claims priority to U.S. Provisional Application Ser. No. 63/389,422 filed Jul. 15, 2022, the disclosure of which is expressly incorporated herein by reference.

### BACKGROUND

Transcranial Magnetic Stimulation (TMS) is a non-invasive neuromodulation treatment method and an FDA-cleared approach to stimulate the brain for treating depression, OCD, and smoking cessation (with many other clinical applications under investigation). A TMS system includes an electric pulse generator or stimulator that is connected to a magnetic coil that is connected to the scalp to generate a varying magnetic field via electromagnetic induction to cause an electric current at a specific area of the brain. TMS over the motor cortex, at sufficient machine power, can cause a thumb/hand movement.

There is nevertheless no easily observable method to determine if the power (or “dose”) is sufficient over treatment areas, such as the prefrontal cortex for depression.

### SUMMARY

An exemplary system and method are disclosed comprising a functional Near-Infrared Spectroscopy (fNIRS) integrated with TMS, collectively referred to as a TMS-fNIRS system configured to evaluate the sufficiency and/or cortical excitability of TMS treatment (e.g., machine power) to reach the brain cortex, e.g., more accurately calibrate the TMS dose to the prefrontal cortex. The TMS-fNIRS system may also provide an assessment as to the sufficiency of location of stimulation, frequency of stimulation, duration of stimulation that can be used to adjust these stimulation parameters. The TMS-fNIRS system may also be used to monitor the effectiveness of brain changes (e.g., moderator and mediators) in view of a TMS-associated stimulation or therapy as well as stopping criteria for the treatment.

The exemplary fNIRS system is configured to non-invasively measure the hemodynamic response, including blood flow and oxygenation, to assess cortical brain function. The exemplary fNIRS system and method include an fNIRS source/detectors that are placed, e.g., under, around, and contralateral to the TMS coil to measure the brain effect of TMS and adjust the power (“dose”) for individual patients to ensure adequate treatment.

The exemplary fNIRS system can provide feedback or visualization to adjust for the coil to cortex distance differences, gyms orientation differences as well as cortical excitability differences between the motor cortex and dorsolateral prefrontal cortex. The transcranial magnetic stimulation (TMS) devices integrated with functional near-infrared spectroscopy (fNIRS) can be used to guide personalized parameters and real-time measurement of target engagement. In some implementations, the feedback or visualization can be used to adjust the location of stimulation, the frequency of stimulation, and/or the duration of stimulation.

In some implementations, the TMS-fNIRS can be used to personalize the treatment of depression or provide other therapeutic effect to improve effectiveness. Depression, especially treatment-resistant depression (TRD), is a growing public health crisis. Although transcranial magnetic stimulation (TMS) is an effective treatment for some patients with TRD, a significant portion does not respond to current therapy. Previous work has demonstrated that adjusting the dose (i.e., the intensity of TMS pulses) can, in certain patient groups (i.e., older), improve clinical outcomes.

In some aspects, the techniques described herein relate to a method including: measuring, via a functional Near-Infrared Spectroscopy (fNIRS) device, a first set of measurements of a treatment region of a patient; performing, via a transcranial magnetic stimulation (TMS) device, TMS treatment to the treatment region for therapeutic effect; measuring, via the fNIRS device, a second set of measurements of the treatment region; determining, by a processor, an applied dosing of the TMS treatment based on at least one of the first set of measurements or the second set of measurements; and outputting control feedback to the TMS device or visualization to a display to adjust one or more parameters or localization of the TMS treatment based on the applied dosing.

In some aspects, the techniques described herein relate to a method, wherein the adjustment includes a coil to treatment region distance adjustment.

In some aspects, the techniques described herein relate to a method, wherein the adjustment includes a gyms orientation adjustment.

In some aspects, the techniques described herein relate to a method, wherein the treatment region is the prefrontal cortex and the adjustment includes a cortical excitability output to a motor cortex and dorsolateral prefrontal cortex.

In some aspects, the techniques described herein relate to a method, wherein the adjustment includes a location of the TMS treatment.

In some aspects, the techniques described herein relate to a method, wherein the adjustment includes a frequency of the TMS treatment.

In some aspects, the techniques described herein relate to a method, wherein the adjustment includes a duration of the TMS treatment.

In some aspects, the techniques described herein relate to a method, wherein the control feedback to the TMS device or visualization to the display is used to monitor an effectiveness of treatment region changes based on the TMS treatment.

In some aspects, the techniques described herein relate to a method, wherein the effectiveness of treatment region changes includes monitoring changes to one or more of moderator or mediators.

In some aspects, the techniques described herein relate to a method, wherein the control feedback to the TMS device or visualization to the display is used to determine a stopping criteria for the TMS treatment.

In some aspects, the techniques described herein relate to a method, wherein the TMS device is configured to generate at least one of: a 12-block sequence of 1 Hz stimulation with 4 sec on and off 26 sec, a 12-block sequence of 10 Hz stimulation with 4 sec on and off 26 sec; or a 48-jittered single pulses stimulation sequence.

In some aspects, the techniques described herein relate to a method, wherein the fNIRS device includes a plurality of light sources and a plurality of light detectors, at least one of each forming a pair, wherein each pair is configured to be placed a predetermined distance apart from one another.

In some aspects, the techniques described herein relate to a method, wherein the TMS treatment is for treatment of depression, the treatment region is the prefrontal cortex, and wherein the control feedback to the TMS device or visualization to the display is to provide sufficient cortical excitability for depression treatment.

In some aspects, the techniques described herein relate to a system including: a functional Near-Infrared Spectroscopy (fNIRS) device; a transcranial magnetic stimulation (TMS) device; and an integrated component having a processor and a memory having instructions stored thereon, wherein execution of the instructions by the processor causes the processor to: receive a first set of fNIRS measurements of a treatment region in response to a TMS treatment applied by the TMS device to the treatment region of a patient; receive a second set of fNIRS measurements of the treatment region; determine an applied dosing of the TMS treatment based on one or more of the first set of fNIRS or the second set of fNIRS; and provide control feedback to the TMS device or visualization to a display to adjust one or more parameters or localization of the TMS treatment based on the applied dosing.

In some aspects, the techniques described herein relate to a system, wherein the instruction further causes the processor to provide control feedback to the TMS device or visualization to the display to adjust one or more of a coil to treatment region distance, a gyrus orientation, an excitability output, a location of the TMS treatment, a frequency of the TMS treatment, a duration of the TMS treatment, an effectiveness of treatment region changes based on the TMS treatment, or a stopping criteria for the TMS treatment.

In some aspects, the techniques described herein relate to a system, wherein the TMS device is configured to generate at least one of: a 12-block sequence of 1 Hz stimulation with 4 sec on and off 26 sec, a 12-block sequence of 10 Hz stimulation with 4 sec on and off 26 sec; or a 48-jittered single pulses stimulation sequence.

In some aspects, the techniques described herein relate to a system, wherein the fNIRS device includes a plurality of light sources and a plurality of light detectors, at least one of each forming a pair, wherein each pair is configured to be placed a predetermined distance apart from one another.

In some aspects, the techniques described herein relate to a non-transitory computer-readable medium for a transcranial magnetic stimulation (TMS) functional Near-Infrared Spectroscopy (fNIRS) (TMS-fNIRS), system the non-transitory computer-readable medium having instructions stored thereon, wherein execution of the instructions by one or more processors causes the one or more processors to: receive a first set of fNIRS measurements from a fNIRS device of the TMS-fNIRS system of a treatment region in response to a TMS treatment applied by a TMS device of the TMS-fNIRS system to the treatment region of a patient; receive a second set of fNIRS measurements from the fNIRS device of the treatment region; determine an applied dosing of the TMS treatment based on one or more of the first set of fNIRS or the second set of fNIRS; and provide control feedback to the TMS device or visualization to a display to adjust one or more parameters or localization of the TMS treatment based on the applied dosing.

In some aspects, the techniques described herein relate to a non-transitory computer-readable medium, wherein the instruction further causes the processor to provide control feedback to the TMS device or visualization to the display to adjust one or more of a coil to treatment region distance, a gyms orientation, an excitability output, a location of the TMS treatment, a frequency of the TMS treatment, a duration of the TMS treatment, an effectiveness of treatment region changes based on the TMS treatment, or a stopping criteria for the TMS treatment.

In some aspects, the techniques described herein relate to a non-transitory computer-readable medium, wherein the TMS treatment is for treatment of depression, the treatment region is the prefrontal cortex, and wherein the control feedback to the TMS system or visualization to the display is to provide sufficient cortical excitability for depression treatment.

For a more complete understanding of the present disclosure, reference is now made to the following brief description, taken in connection with the accompanying drawings and detailed description, wherein like reference numerals represent like parts.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 shows an example transcranial magnetic stimulation (TMS) device integrated with functional near-infrared spectroscopy (fNIRS) (TMS-fNIRS) system according to various aspects of the disclosure.

FIG. 2A shows an example configuration of the TMS-fNIRS system of FIG. 1 according to various aspects of the disclosure.

FIG. 2B shows another example configuration of the TMS-fNIRS system of FIG. 1 according to various aspects of the disclosure.

FIG. 3 shows an example method to operate a TMS-fNIRS system of FIG. 1 according to various aspects of the disclosure.

FIG. 4 shows averaged time course of HbO₂ signals for the MC and the dl-PFC according to various aspects of the disclosure.

FIG. 5 illustrates an exemplary computer system suitable for implementing the several aspects of the disclosure.

### DETAILED DESCRIPTION

Each and every feature described herein, and each and every combination of two or more of such features, is included within the scope of the present invention, provided that the features included in such a combination are not mutually inconsistent.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

### Example System

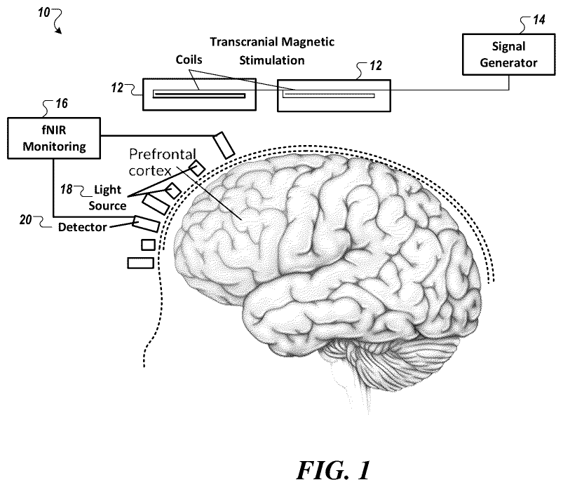

FIG. 1 shows an example TMS-fNIRS system 10. In the example shown in FIG. 1, the TMS-fNIRS system 10 includes (i) one or more transcranial magnetic stimulation (TMS) coils 12 (in the example shown, two coils 12 are used, though any number of coils may be used) that are connected to a signal generator 14, sometimes referred to as an electric pulse generator or stimulator and (ii) an fNIRS system 16 comprising a set of probes that are placed on the scalp of a patient. While a particular orientation of the coils 12 and the fNIRS system 16 is shown relative to each other and the brain of the patient, this example is non-limiting. Generally, the fNIRS system 16 may be placed under, around, and/or contralateral to the TMS coils 12. The fNIRS system 16 and the coils 12 may be placed anywhere on the scalp of the patient to stimulate excitability in a desired region(s) of the brain.

The stimulation generated by the signal generator 14 and delivered by the TMS coils 12 may include (1) 12 blocks of 1 Hz stimulation with 4 sec on and off 26 sec; 100% MT; 48 pulses; (2) 12 blocks of 10 Hz stimulation with 4 sec on and off 26 sec; 100% MT; 480 pulses; (3) 48 jittered single pulses stimulation; using 100% MT; 48 pulses; (4) 48 jittered single pulses stimulation; using 120% MT; 48 pulses. The Total time once stimulation starts could be 62 minutes, as one example.

In some embodiments, the example TMS-fNIRS system 10 is configured as a 1-Hz to 10-Hz FNIRS-TMS. Examples of transcranial magnetic stimulation devices are described in U.S. Pat. Nos. 10,004,915; 9,682,249; and 10,112,056.

Functional near-infrared spectroscopy (fNIRS) system 16 is configured to perform optical brain monitoring using near-infrared spectroscopy for functional neuroimaging. Using the fNIRS system 16, the brain activity is measured by using near-infrared light to estimate cortical hemodynamic activity, which can occur in response to neural activity.

In the example shown in FIG. 1, the fNIRS system 16 includes a set of light sources 18 and a set of light detectors 20. In an example, the light sources 18 and light detectors could be 3 centimeters apart from one another. Other distances or spacing between the light sources 18 and the light detectors 20 may be used.

fNIRS may be placed over the ipsilateral PFC (ipsiPFC) as well as the contralateral prefrontal cortex (contraPFC). The ratio of the ipsilateral PFC and the contralateral prefrontal cortex measurements could be determined and used as an assessment for depression.

TMS-fNIRS System #1

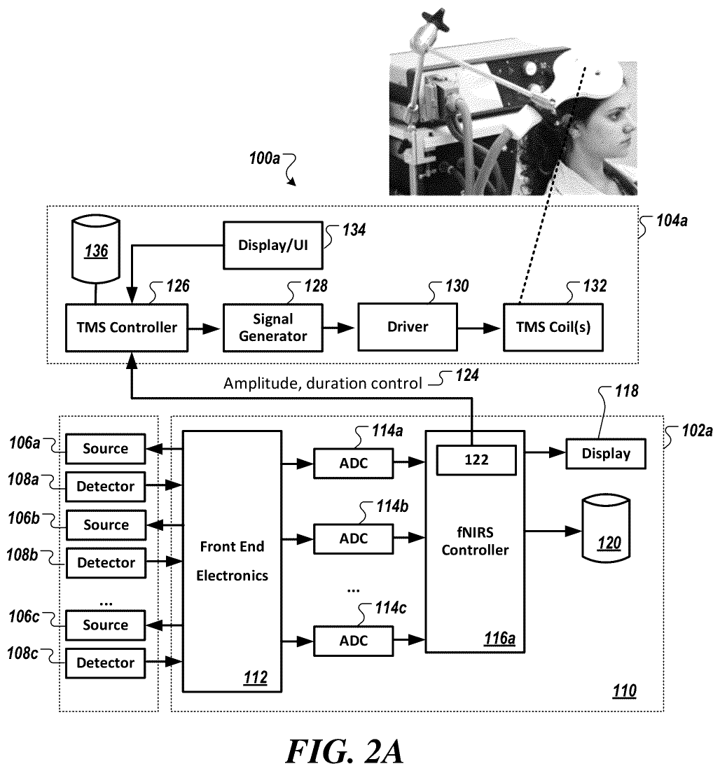

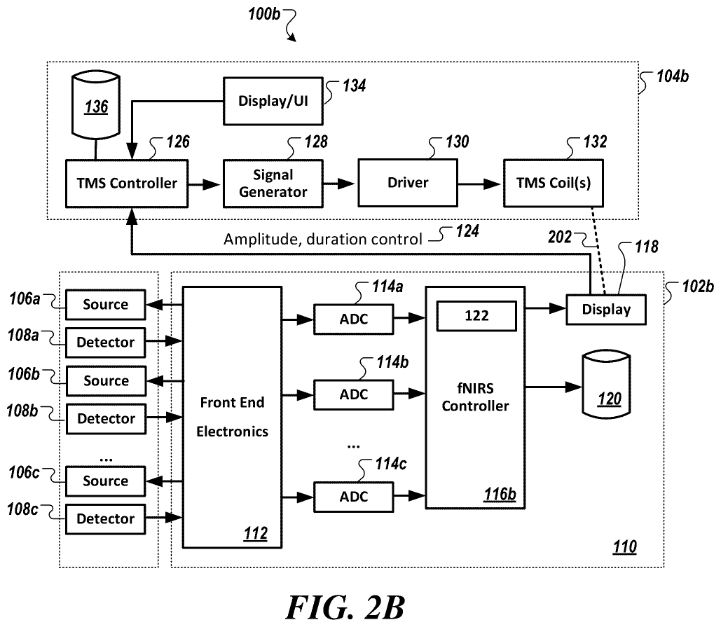

FIG. 2A shows an example configuration of the TMS-fNIRS system of FIG. 1 in accordance with an illustrative embodiment. In the example shown in FIG. 2A, the TMS-fNIRS system 100 (shown as 100a) includes an fNIRS system 102 (shown as 102a) and a TMS system 104 (shown as 104a).

The fNIRS system 102a is configured to evaluate the sufficiency and/or cortical excitability of TMS treatment (e.g., machine power) to reach the brain cortex, e.g., more accurately calibrate the TMS dose to the prefrontal cortex. In the example shown in FIG. 2A, the fNIRS system 102a includes a set of light sources 106 (shown as 106a, 106b, 106c) and detectors 108 (shown as 108a, 108b, and 108c), e.g., that are positioned on a cap to be placed on a patient. In the example shown, at least one of each of the set of light sources 106 and detectors 108 form a pair, where each pair of the light sources and detectors (e.g., 106, 108) are positioned a predetermined distance apart (e.g., 3 cm). The set of light sources and detectors (e.g., 106, 108) may be positioned to interrogate signals in the prefrontal cortex region of the patient. Different caps may be provided for different scalp sizes. The set of light sources and detectors (e.g., 106, 108) may vary in number of sources and detectors from 1 each to 32 each, for example. In a specific example, the set of light sources and detectors (e.g., 106, 108) is an array of 16×16.

The set of light sources and detectors (e.g., 106, 108) are connected to a processing system 110 comprising front-end circuitries 112, analog to digital converters 114 (shown as 114a, 114b, and 114c), controller 116 (shown as “fNIRS Controller” 116a), display 118, and data store 120.

The fNIRS system 102a is configured to generate a set of light stimuli to the patient scalp (e.g., at the prefrontal cortex) via the light sources (e.g., 106) and to detect the response from the stimuli via the detectors (e.g., 108). The acquired fNIRS signals are filtered and amplified by the front-end circuitries 112 and converted to a digital signal via the ADCs (e.g., 114). The controller 116a receives the digitized fNIRS signals and executes an operator 122 to determine applied dosing for a given transcranial magnetic brain stimulation treatment that is executing.

The controller 116a can generate a feedback 124 to adjust one or more parameters or localization of the TMS stimulation based on the determined dosing, e.g., to provide sufficient cortical excitability for depression treatment. In some embodiments, the feedback or visualization can be used to adjust the location of stimulation, the frequency of stimulation, the amplitude of stimulation, and/or the duration of stimulation. In some embodiments, the feedback or visualization can be used to monitor the effectiveness of brain changes (e.g., moderator and mediators) in view of a TMS-associated stimulation or therapy as well as stopping criteria for the treatment.

In the example shown in FIG. 2A, the TMS system 104a includes a TMS controller 126, a signal generator 128, driver circuitries 130, one or more TMS coils 132, a display/UI 134, and a data store 136. In some embodiments, the TMS system 104a is configured as a handheld device or device-mounted device having a front region that houses the set of one or more TMS coils 132. The data store 136 includes a set of pre-defined parameters and instruction sets to perform a treatment.

The TMS controller 126 may interface with the data store 136 to obtain a set of parameters and instruction sets to perform a given treatment via a selection made through the display/UI 134. The set of parameters and instruction sets of the controller 126 may direct the signal generator 128 to generate a set of sequences. An example sequence includes 12 blocks of 1 Hz stimulation with 4 sec on and off, 26 sec; 100% MT; 48 pulses. Another example sequence includes 12 blocks of 10 Hz stimulation with 4 sec on and off 26 sec; 100% MT; 480 pulses. Another example sequence includes 48 jittered single pulses stimulation, using 100% MT. Yet another example sequence includes 48 jittered single pulses stimulation, using 120% MT, 48 pulses. The pre-defined parameters may include waveform shape (e.g., rectangular, sinusoidal, triangular), amplitude, a number of blocks, frequency of stimulation duty cycle (percentage one and percentage off), and total stimulation time, among others.

The signal generator 128 may output the waveform sequence to the driver circuitries 130, e.g., comprising isolation circuitries and coil amplifiers. The driver circuitries 130 may output the TMS coil signal to the TMS coil(s) 132. The generated magnetic field may be in the range of an MRI (e.g., 1T, 2T, 3T, among others), and the pulse generally reaches no more than 5 centimeters into the brain, though stimulation may be employed with focused coils to shape the generated magnetic field. From the Biot-Savart law, per the equation below:

$$B = \frac{{μ}_{0}}{4 π} I {\int }_{C} \frac{d 1 \times \hat{r}}{{r}^{2}} ,$$

in which a current I through one of the TMS coils 132 can generate a magnetic field B around that coil. The transcranial magnetic stimulation circuit (e.g., 128, 130) may quickly discharge current from a large capacitor (e.g., in 130) into the TMS coil 132 to produce pulsed magnetic fields, e.g., between 2 and 3 Tesla in strength.

While the example TMS-fNIRS system 100 is described above as producing TMS signals, it is contemplated that the signal generator 128, driver circuitry 130, and TMS coils 132 may likewise operate to produce theta burst stimulation (TBS). Therefore, throughout this disclosure, references to TMS may likewise refer to TBS. Throughout this disclosure, any area of the body targeted for treatment by TMS or TBS is referred to as a treatment region, including any region of the brain (e.g., prefrontal cortex, parietal, temporal, etc.), the spinal cord, peripheral nerves, or any other region of the body responsive to such treatment.

TMS-fNIRS System #2

FIG. 2B shows another example configuration of the TMS-fNIRS system 100 (shown as 100b) of FIG. 1 in accordance with an illustrative embodiment.

The TMS-fNIRS system 100b includes a fNIRS system 102 (shown as 102b) and a TMS system 104 (shown as 104b).

The fNIRS system 102b is also configured to evaluate the sufficiency and/or cortical excitability of TMS treatment (e.g., machine power) to reach the brain cortex, e.g., more accurately calibrate the TMS dose to the prefrontal cortex. In the example shown in FIG. 2B, the fNIRS system 102b includes a set of light sources 106 (shown as 106a, 106b, 106c), detectors 108 (shown as 108a, 108b, and 108c), e.g., that are positioned on a cap to be placed on a patient, as well as associated circuitries (e.g., 112, 114, 116), e.g., as described in relation to FIG. 2A.

The fNIRS system 102b is configured to generate a set of light stimuli to the patient scalp (e.g., at the prefrontal cortex) via the light sources (e.g., 106) and to detect the response from the stimuli via the detectors (e.g., 108). The acquired fNIRS signals are filtered and amplified by the front-end circuitries 112 and converted to a digital signal via the ADCs (e.g., 114). The controller 116b receives the digitized fNIRS signals and executes an operator 122 to determine applied dosing for a given transcranial magnetic brain stimulation treatment that is executing.

The controller 116b can generate a visualization of the applied dosage and present the visualization on display 118 to allow the operator (e.g., technician) to adjust one or more parameters or localization via feedback 124 to the TMS stimulation based on the determined dosing, e.g., to provide sufficient cortical excitability for depression treatment. The operator can also adjust 202 the orientation and/or position of the TMS coils 132 with respect to the patient based on the visualization.

In alternative embodiments, the parameters 124 may be modified by the fNIRS controller 116b.

### Example Method

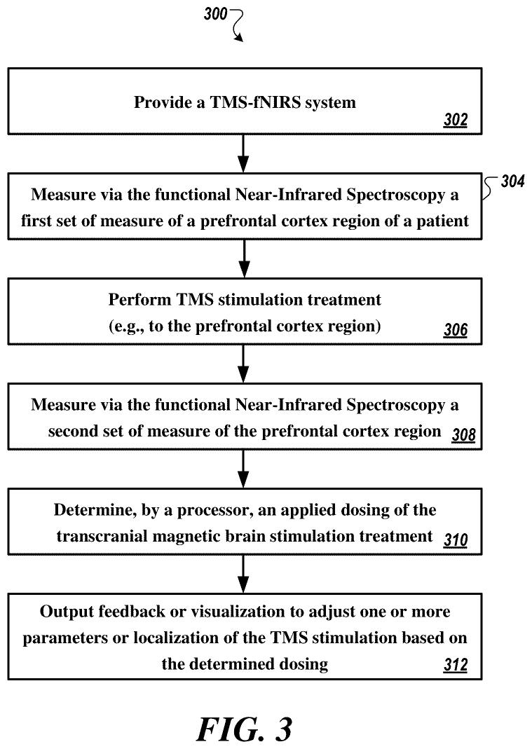

FIG. 3 shows an example method to operate a TMS-fNIRS system such as TMS-fNIRS systems 100a, 100b described above.

Method 300 includes providing (302) a TMS-fNIRS system comprising a functional Near-Infrared Spectroscopy (fNIRS) device and a transcranial magnetic stimulation (TMS) device.

Method 300 includes measuring (304), via the functional Near-Infrared Spectroscopy, a first set of measures of a prefrontal cortex region of a patient.

Method 300 includes performing (306) TMS stimulation treatment to the prefrontal cortex region (e.g., to treat depression). The TMS stimulation treatment may include generating a 12-block sequence of 1 Hz stimulation with 4 seconds on and off 26 seconds, a 12-block sequence of 10 Hz stimulation with 4 seconds-on and off 26 seconds; or a 48-jittered single pulses stimulation sequence.

Method 300 includes measuring (308), via the functional Near-Infrared Spectroscopy, a second set of measures of the prefrontal cortex region.

Method 300 includes determining (310), by a processor, applied dosing of the transcranial magnetic brain stimulation treatment.

Method 300 includes outputting (312) feedback or visualization to adjust one or more parameters or localization of the TMS stimulation based on the determined dosing, e.g., to provide sufficient cortical excitability for depression treatment. In some embodiments, the adjustment may include adjusting a coil to cortex distance, a gyms orientation, and/or a cortical excitability output (e.g., parameters to the TMS controller) to the motor cortex and dorsolateral prefrontal cortex. In some embodiments, the feedback or visualization can be used to adjust the location of stimulation, the frequency of stimulation, and/or the duration of stimulation. In some embodiments, the feedback or visualization can be used to monitor the effectiveness of brain changes (e.g., moderator and mediators) in view of a TMS-associated stimulation or therapy as well as stopping criteria for the treatment.

While the method 300 is described above with the example of treatment of the prefrontal cortex region for treatment of depression, it is understood that different regions of the brain may be treated with the same or different stimulation patterns, waveforms, amplitudes, frequency, and/or durations for treatment of depression or other conditions. Likewise, the same or different stimulation patterns, waveforms, amplitudes, frequency, and/or durations for treatment may be used in the prefrontal cortex or other regions of the brain for treatment of conditions other than depression, such as generalized anxiety disorder, posttraumatic distress disorder, and/or chronic pain or otherwise providing therapeutic effect. More generally, the method 300 may be applied to any area of the brain (e.g., parietal, temporal, etc.) that do not have clear behavioral outputs. Moreover, it is contemplated that the method 300 may be used to treat other areas of the body other than the brain, such as to stimulate the spinal cord and/or peripheral nerves.

### Experimental Results and Example

A study may be conducted to develop the best protocol to interrogate cortical excitability that provides a statistically significant change that is reliable. The dose-finding aspect would facilitate the development of a protocol that is the least burden to be adopted. The clinical trial portion would also facilitate a preliminary assessment of the approach that would inform future clinical trials. Participants with TRD who are candidates for TMS may be recruited, consented, assessed, have a structural MRI, and then undergo TMS-fNIRS

fNIRS recording and processing A multichannel continuous-wave fNIRS imaging system (NIRx Orlando, FL) may be used to measure TMS-related cortical response. This instrument may include 16 light sources (each emitting laser light of 760 nm and 850 nm) and 16 detectors (one detector may be split for 8 short-distance detectors) that are attached to a headgear via optical fibers. The sources and detectors for the channel of interest to measure brain changes may be located 3 cm apart. The geometrical layout of these optodes for illumination and detection may cover the MC and dl-PFC regions bilaterally.

The short-distance channels may enable the contribution of signals from superficial structures (e.g., scalp) to be eliminated from the brain channels. The headgear placement may be consistent across different participants and scanning sessions. Thus, the cortical activity can be quantified and monitored longitudinally in each optical channel for each subject. To achieve this, the position of all optodes may be digitized with respect to selected anatomical landmarks such that the placement of optical channels can be registered on a standard head and brain atlas prior to each measuring session; (Tools like fOLD (44) or Array Designer (45) can be used for this purpose if needed [46-48]). After optical signals are recorded during TMS sessions according to the protocol for selected paradigms as specified above, a MATLAB® (Mathworks, Inc., Natick, MA) pipeline primarily built upon Homer3 [49] and AnalyZIR [50] may be used for fNIRS data processing and analytics [50-57]. Specifically, the signal quality of the collected raw intensity may be checked for channel pruning using the conservative method PHOEBE [58]. After conversion from raw intensity to optical density, motion artifacts may be removed using selected motion correction algorithms (e.g., wavelet decomposition [59], tPCA[60]). Detrending and physiological noise removal may be performed using appropriate techniques (i.e., linear regression and bandpass filtering, respectively). For the purpose of conversion from optical density to oxy- and deoxyhemoglobin concentration changes, the modified Beer-Lambert law [61] may be employed to account for the age-dependent differential path length factor [62]. Significantly activated channels can be determined after fitting the general linear model (GLM) to HbO2 and Hb concentration changes over time [50, 52], and additional analytics (e.g., group-level analysis [50], connectivity analysis [63], functional data analysis [64]) may also be performed when necessary.

Although Transcranial Magnetic Stimulation (TMS) is a highly effective treatment for patients with difficult-to-treat depression, improvement in remission rates is critically needed. The fundamental problem is that TMS is dosed over the motor cortex, which has an observable functional measure (e.g., finger twitch); however, the treatment is performed over the prefrontal cortex (i.e., no observable change). The study employs a functional Near-Infrared Spectroscopy (fNIRS) integrated with TMS (TMS-fNIRS) to personalize the treatment dosing of TMS more accurately over the prefrontal cortex by more accurately calibrating the dose to the prefrontal cortex. The system is configured to adjust for the coil to cortex distance differences, gyrus orientation differences as well as cortical excitability differences between the motor cortex and dorsolateral prefrontal cortex.

Significant fNIRS signal at the individual level with TMS over the motor cortex: TMS can be used to stimulate the brain over the motor cortex while fNIRS can simultaneously measure the brain effect (i.e., TMS-fNIRS). The TMS-fNIRS test is configured to determine a quantifiable measure of motor cortex brain activity at a stimulation intensity that has been determined by an observable behavioral output to activate the motor circuit (i.e., motor threshold—MT). The test may employ various paradigms to determine which produces the most robust signal at the individual level in participants who would be eligible for clinical TMS of depression, e.g., the intensities of stimulation (100% MT v. 120% MT), pulse numbers (48 v. 480), and frequency (1 Hz v. 10 Hz).

Significant fNIRS signal at the individual level with TMS over the prefrontal cortex: The TMS-fNIRS test can determine a statistically significant signal measured over the dorsolateral prefrontal cortex. The TMS-fNIRS test may determine a quantifiable measure of prefrontal cortical brain activity at a stimulation intensity determined by MT for an individual. The study may test the same above paradigms to determine which produces the most robust signal at the individual level in participants who would be eligible for clinical TMS to treat depression.

An R61 study may be conducted to evaluate TMS/fNIRS protocols to provide the best TMS/fNIRS testing paradigm to be used in the R33 study. In some embodiments, dysphoria symptomatic patients may be evaluated.

The R33 portion may be used for preliminary testing of the clinical impact of the relationship between the magnitude of the TMS-fNIRS signal of the motor cortex as compared to the prefrontal cortex. This may provide the foundation for a clinical trial testing the clinical utility of this measure for adjusting the dose of TMS in the treatment of depression.

Overview—The R61 portion of the study is configured to determine an optimal TMS/fNIRS protocol to obtain a magnitude measure of cortical stimulation with enough signal to noise to be reliable. The Go/No-Go criteria are based on a reliable fNIRS signal over the motor cortex and the prefrontal cortex for individual participants being acquired to calibrate prefrontal TMS dose. Dose finding may be used to determine whether the entire protocol is needed or an abbreviated version achieves the same level of quality.

R61 Protocol. Participants aged 18-60 years old may be recruited from the community. These clinics serve a very diverse population in the greater Tallahassee region. The study may recruit participants that would be eligible to receive TMS treatment, including having a diagnosis of MDD that has not responded to one adequate antidepressant or intolerance of four antidepressants and no medical contraindications for TMS. Participants may have all treatments stable for the duration of their enrollment in the study.

Visit 1 may involve obtaining consent after all questions of participants are answered. Demographic and clinical information may be obtained. Clinical information may include a psychiatric interview that includes an assessment for safety (i.e., suicide and violence risk, TMS seizure risk), current psychiatric diagnoses, clinical rating scales, patient-rated scales, and urine drug and pregnancy screen. Clinician-rated scales may include the MADRS and Columbia Suicide Scale. The patient-rated scales may include the PHQ-9, GAD-7, PCL-5, SF-36, and pain scale.

Visit 2 may involve obtaining a T1w MPRAGE brain scan sagittally acquired (176 1 mm slices) in-plane matrix of 256×256 at 1×1 mm, TR 2.5 s, TI 1.06 s, flip angle 8 degrees. This sequence has been used in multiple other protocols in our center with good image quality.

Visit 3 may start with confirming no changes in safety considerations. The fNIRS optodes may be placed on the participants' scalp and adjusted to achieve maximal signal to noise. Using Brainsight, the external head landmarks and fNIRS optodes may be co-registered with the participant's structural MRI. Next, the location and dose may be determined for the Motor Threshold (MT). The location may be recorded in Brainsight. Participants may then have their order of four stimulation paradigms (see below) randomized. The first TMS/fNIRS protocols may be performed over the Motor Cortex (MC), and then after a 10-minute break, the next protocol in the randomization may be performed. This may be repeated until all four protocols have been completed. The coil may then be moved forward over the dorsolateral prefrontal cortex (dl-PFC) as defined by the Beam F3 location and position recorded by Brainsight. The same protocols as over the MC may be followed for the dl-PFC.

The four protocols may each be a total of 8 minutes. There may be a one-minute pre-stimulation baseline with the subject sitting quietly with eyes open and a one-minute post-stimulation baseline with the subject sitting quietly with eyes open. There may be a 10-minute break between each TMS/fNIRS protocol. Stimulation protocol parameters: (1) 12 blocks of 1 Hz with 4 sec on and off 26 sec; 100% MT; 48 pulses; (2) 12 blocks of 10 Hz with 4 sec on and off 26 sec; 100% MT; 480 pulses; (3) 48 jittered single pulses; using 100% MT; 48 pulses; (4) 48 jittered single pulses; using 120% MT; 48 pulses. Total time once starts stimulation maybe 62 minutes for testing over MC followed by a 20-minute break and then 62 minutes over dl-PFC.

The data may be checked for the quality of each channel.

The study may assess for the magnitude of signal to noise for each protocol at the individual level. The study may assess the signal dose-response of each rTMS/fNIRS protocol.

R33 Protocol. Participant recruitment and inclusion/exclusion may be the same, except that participants must not only be eligible to receive TMS but may be treated with TMS for their MDD.

Visit 1 may involve obtaining consent after all questions are answered and demographic and clinical information obtained. Clinical information may consist of a psychiatric interview, including an assessment for safety (i.e., suicide and violence risk, TMS seizure risk), current psychiatric diagnoses, clinical rating scales, patient-rated scales, and urine drug and pregnancy screen. Clinician-rated scales may include the MADRS and Columbia Suicide Scale. The patient-rated scales may include the PHQ-9, GAD-7, PCL-5, SF-36, and pain scale. Entry criteria may be PHQ-9>10 may, and the primary endpoint may be based on MADRS.

Visit 2 may involve obtaining the same structural MRI sequence as in the R61.

Visit 3 may start with confirming no changes in safety considerations. The fNIRS optodes may be placed on the participants' scalp and adjusted to achieve maximal signal to noise. Using Brainsight, the external head landmarks and fNIRS optodes may be co-registered with the participant's structural MRI. Next, the location and dose may be determined for the Motor Threshold (MT). The best TMS-fNIRS protocol from the R61 may be performed over the Motor Cortex and then, after a 10-minute break, the same protocol over the PFC. After TMS-fNIRS is completed, the patient may undergo a standard TMS treatment (Beam F3, 120% MT, 10 Hz, 4 sec on and 12 sec off, for 3000 pulses).

Visits 4-6, 8-31 may be standard clinical TMS treatment visits.

Visits 7, 32: Prior to starting TMS treatments, the TMS/fNIRS procedures may be repeated. At the completion of Visit 32 the participant may be referred to clinical care.

The magnitude of brain change with TMS/fNIRS over ipsilateral PFC (ipsiPFC) compared to the contralateral prefrontal cortex (contraPFC) may be used as a treatment outcome in TMS for depression. The contraPFC/ipsiPFC ratio, as measured by the exemplary TMS-fNIRS system, may be positively correlated with depression treatment outcomes as measured by MADRS.

Use of SIMB as covariate may be used. Methods for adjustment of PFC dose may be performed to match the degree of activation over MC. The method may determine the minimal contralateral activation and adjusting the dose/location of the ipsilateral to achieve a threshold level of activation.

Discussion

Depression is a major international public health crisis that has resulted in a significant burden of disease and costs to society. The World Health Organization (WHO) estimates that over 300 million or 4.4% of the world's population are suffering from depression. Depression is the single largest contributor to non-fatal health loss (2017). The economic costs of Major Depressive Disorder in the U.S. have been estimated at $210.5 billion (Greenberg et al., 2015). The financial and individual impact is much greater for those suffering from treatment-resistant depression (TRD) (Amos et al., 2018; Pilon et al., 2019), which is typically defined as having failed at least two adequate antidepressant medication treatment trials (Gaynes et al., 2020).

There are a number of effective treatments for Major Depressive Disorder, such as pharmacotherapy and psychotherapy, but a significant portion of patients continue to struggle with depressive symptoms even after many full courses of treatment. Transcranial magnetic stimulation (TMS) is a neuromodulation treatment that has demonstrated efficacy [6], [7] and effectiveness for MDD [8,9] including in treatment-resistant populations with multiple co-morbidities [11,12]. Although highly effective for many patients, improvement in remission rates are still critically needed. A significant portion of treated patients continues to have disabling depressive symptoms even after a complete course of TMS.

One of the earliest findings in the field of TMS is that differences in distance from coil to cortex over the motor cortex compared to over the prefrontal cortex is an important treatment variable [13-17]. As patients age, the distance from the coil to the cortex over the prefrontal cortex increases more rapidly than the distance over the motor cortex. The fundamental problem is that TMS is dosed using the motor cortex, which has an observable functional measure (e.g., finger twitch); however, the treatment is typically performed by stimulating the prefrontal cortex (i.e., no easily observable change). This recognition of an important treatment variable led to adjustments in treatment parameters that resulted in older age going from a negative predictor of outcome to a positive predictor [10,11]. In addition to differences in coil-to-cortex differences that can now be adjusted by obtaining an MRI and calculating adjustment in stimulation intensity, there are also differences in gyri orientation as well as a potential degree of cortical excitability between the two brain regions. Caufield et al. recently demonstrated using electric field (E-field) modeling that the standard 120% MT dose was often, but not always inadequate to get the equivalent stimulation dose of the MC to the dl-PFC. In addition, the Caufield et al. study demonstrated the important finding that there is considerable individual variability between patients [19]. Thus, adjusting for the individual differences between patients in order to personalize the TMS dose is needed.

Functional near-infrared spectroscopy (fNIRS) is an optical imaging technology that offers a method to investigate brain changes associated with TMS that has some unique beneficial properties [20-22]. It can measure the light attenuation of the biological tissues in the near-infrared spectrum (670-900 nm). Near-infrared light penetrates tissues to a depth of several centimeters and is mainly absorbed by two principal chromophores in blood flow: oxygenated hemoglobin (HbO2) and deoxygenated hemoglobin (Hb). Therefore, by measuring the change of light attenuation from a baseline stage at two or more wavelengths, the changes in HbO2 and Hb concentrations in the tissues can be quantified [22]. fNIRS can measure functional brain changes in the superficial cortex (approximately 1 cm square) at a frequency of 10 Hz (ten data points every second). fNIRS offers the potential to measure immediate brain effects of TMS in areas of the brain that do not have an immediate and quantifiable behavioral change [23]. Several studies have demonstrated support for this idea. Allen et al. demonstrated in an animal model that optical imaging of hemodynamic changes provides an effective means to monitor neuronal changes from TMS. Additionally, several investigators have employed optical imaging to study brain changes associated with single-pulse and repetitive TMS [23, 25-28]. Optical imaging has also been shown to be a reliable measure at the level of an individual (versus group averaged) [29]. Thus, fNIRS has several unique properties that make it a good candidate for integrating with TMS in order to increase the personalization of the dose with the goal of improving treatment outcomes.

In contrast, the exemplary TMS-fNIRS system is configured to personalize treatment dosing more accurately in other regions of the prefrontal cortex, e.g., by adjusting for: 1) coil to cortex distance differences; 2) gyri orientation differences as well as; 3) cortical excitability differences between the motor cortex and dorsolateral prefrontal cortex. Adjusting for cortical excitability cannot be accomplished with E-field modeling. These adjustments could further increase the personalization of care for patients with TRD, a devastating disorder by any standard. In addition, the measurement of change in cortical activation from TMS from one location to another may serve as a measure of the ability of TMS to adequately activate the cortical surface. Previous adjustments in dose have resulted in improved clinical outcomes. TMS-fNIRS could be deployed rapidly in current clinical TMS clinics without expensive tests or the additional patient burden of obtaining an MRI.

Previous used brain imaging can be used in a unique manner and integrated with TMS for adjusting the dose of the TMS treatments. TMS involves brain stimulation utilizing the electromagnetic spectrum, while fNIRS utilizes the properties of light to measure brain activity. Thus, there is no direct interference of the energy spectrums of the stimulation and measuring. TMS-fNIRS could provide a much great personalization of treatment than simply determining the dose over the motor cortex as is done in standard clinical practice.

This technique, although being developed in the use of TMS treatment of MDD, could be critical for TMS research or clinical treatments that involves determining dose over the motor cortex and then stimulating in a different brain region. Similar to using the distance from coil to cortex to adjust the dose, the further refinement of the dose could result in a great proportion of patients experiencing a remission from depression who receive TMS.

A study was previously conducted to evaluate the ability to reliably acquire an adequate and reliable signal with TMS/fNIRS in healthy controls (Kozel 2009 et al. Neuroimage).

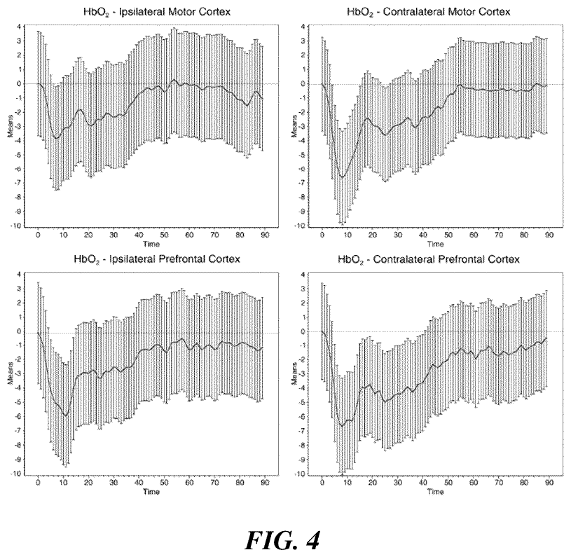

FIG. 4 shows averaged time course of HbO₂ signals for the MC and the dl-PFC according to various aspects of the disclosure. Measurements were made under the coil and in the corresponding contralateral cortex. One hertz stimulation was performed for 10 seconds followed by 80 seconds of rest for 15 epochs. The x-axis represents the time from 0 to 90 seconds in the epoch, and the y-axis represents the mean and 95% confidence interval for HbO2 concentration in μM/L.

Twelve healthy, nonmedicated participants ages 18-50 years were recruited from the local community. They underwent two visits of simultaneous rTMS/fNIRS separated by 2 to 3 days. In each visit, the motor cortex and subsequently the prefrontal cortex (5 cm anterior to the motor cortex) were stimulated (1 Hz, max 120% MT, 10 s on with 80 s off, for 15 trains) while simultaneous fNIRS data were acquired from the ipsilateral and contralateral brain regions. The 11 participants with adequate data (9 male) had a mean age of 31.8 (s.d. 10.2, range 20-49) years. There was no significant difference in fNIRS between Visit 1 and Visit 2. Stimulation of both the motor and prefrontal cortices resulted in a significant decrease in oxygenated hemoglobin (HbO2) concentration in both the ipsilateral and contralateral cortices. The ipsilateral and contralateral changes showed high temporal consistency.

Subsequent analysis demonstrated that the test-retest reliability of the combined TMS/fNIRS assessed using repeated measurements performed two to three days apart demonstrated that the change in HbO2 amplitudes had moderate-to-high reliability at the group level, and the spatial patterns of the topographic images have high reproducibility in size and a moderate degree of overlap at the individual level (Tian 2012 J Biomed Optics). This study was in healthy participants and only assessed one testing paradigm, but it does demonstrate the ability to acquire the data and supports the reliability of the TMS/fNIRS measure for an individual as well as the similarity of fNIRS signal for ipsilateral and contralateral cortical activation with TMS in healthy participants.

### Example Computing System

It should be appreciated that the logical operations for the analysis of the fNIRS using the processes described above can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device 200 to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media (e.g., a non-transitory computer readable medium).

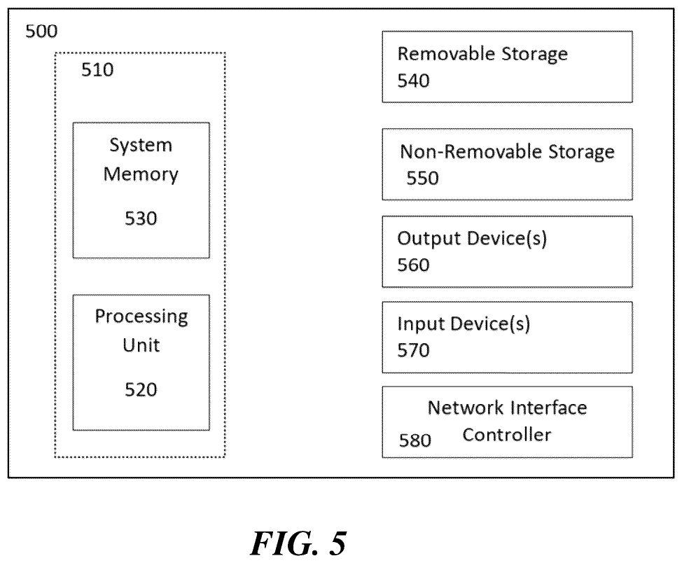

FIG. 5 illustrates an exemplary computer system 500 suitable for implementing the several embodiments of the disclosure. For example, one or more of the fNIRS system 102, TMS system 104, or elements thereof may be implemented as the computer system 500.

It should be appreciated that the logical operations described herein with respect to the various figures may be implemented (1) as a sequence of computer implemented acts or program modules (i.e., software) running on a computing device (e.g., the computing device described in FIG. 5), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts, and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 5, an example computing device 500 upon which embodiments of the invention may be implemented is illustrated. For example, one or more of the fNIRS system 102, TMS system 104, or elements thereof may be implemented as the computer system 500. It should be understood that the example computing device 500 is only one example of a suitable computing environment upon which embodiments of the invention may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In some embodiments, the computing device 500 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In some embodiments, virtualization software may be employed by the computing device 500 to provide the functionality of a number of servers that is not directly bound to the number of computers in the computing device 500. For example, virtualization software may provide twenty virtual servers on four physical computers. In some embodiments, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In its most basic configuration, computing device 500 typically includes at least one processing unit 520 and system memory 530. Depending on the exact configuration and type of computing device, system memory 530 may be volatile (such as random access memory (RAM)), non-volatile (such as read-only memory (ROM), flash memory, etc.), or some combination of the two. This most basic configuration is illustrated in FIG. 5 by dashed line 510. The processing unit 520 may be a standard programmable processor that performs arithmetic and logic operations necessary for operation of the computing device 500. While only one processing unit 520 is shown, multiple processors may be present. Thus, while instructions may be discussed as executed by a processor, the instructions may be executed simultaneously, serially, or otherwise executed by one or multiple processors. The computing device 500 may also include a bus or other communication mechanism for communicating information among various components of the computing device 500.

Computing device 500 may have additional features/functionality. For example, computing device 500 may include additional storage such as removable storage 540 and non-removable storage 550 including, but not limited to, magnetic or optical disks or tapes. Computing device 500 may also contain network connection(s) 580 that allow the device to communicate with other devices such as over the communication pathways described herein. The network connection(s) 580 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 500 may also have input device(s) 570 such as a keyboard, keypads, switches, dials, mice, track balls, touch screens, voice recognizers, card readers, paper tape readers, or other well-known input devices. Output device(s) 560 such as a printer, video monitors, liquid crystal displays (LCDs), touch screen displays, displays, speakers, etc. may also be included. The additional devices may be connected to the bus in order to facilitate communication of data among the components of the computing device 500. All these devices are well known in the art and need not be discussed at length here.

The processing unit 520 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 520 for execution. Example tangible, computer-readable media may include, but is not limited to, volatile media, non-volatile media, removable media, and non-removable media implemented in any method or technology for storage of information such as computer readable instructions, data structures, program modules or other data. System memory 530, removable storage 540, and non-removable storage 550 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

In an example implementation, the processing unit 520 may execute program code stored in the system memory 530. For example, the bus may carry data to the system memory 530, from which the processing unit 520 receives and executes instructions. The data received by the system memory 530 may optionally be stored on the removable storage 540 or the non-removable storage 550 before or after execution by the processing unit 520.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Embodiments of the methods and systems may be described herein with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

In light of the above, it should be appreciated that many types of physical transformations take place in the computer architecture in order to store and execute the software components presented herein. It also should be appreciated that the computer architecture may include other types of computing devices, including handheld computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include the one particular value and/or to the other particular value.

By “comprising” or “containing” or “including” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology may be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.
[1] Depression and Other Common Mental Disorders: Global Health Estimates. Geneva: World Health Organization; 2017.
[2] Greenberg P E, Fournier A A, Sisitsky T, Pike C T, Kessler R C. The economic burden of adults with major depressive disorder in the United States (2005 and 2010). J Clin Psychiatry. 2015; 76(2):155-62. Epub 2015/03/06. doi: 10.4088/JCP.14m09298. PubMed PMID: 25742202.
[3] Amos T B, Tandon N, Lefebvre P, Pilon D, Kamstra R L, Pivneva I, Greenberg P E. Direct and Indirect Cost Burden and Change of Employment Status in Treatment-Resistant Depression: A Matched-Cohort Study Using a US Commercial Claims Database. J Clin Psychiatry. 2018; 79(2). Epub 2018/02/24. doi: 10.4088/JCP.17m11725. PubMed PMID: 29474009.
[4] Pilon D, Joshi K, Sheehan J J, Zichlin M L, Zuckerman P, Lefebvre P, Greenberg P E. Burden of treatment-resistant depression in Medicare: A retrospective claims database analysis. PLoS One. 2019; 14(10):e0223255. Epub 2019/10/11. doi: 10.1371/journal.pone.0223255. PubMed PMID: 31600244; PMCID: PMC6786597
[5] Gaynes B N, Lux L, Gartlehner G, Asher G, Forman-Hoffman V, Green J, Boland E, Weber R P, Randolph C, Bann C, Coker-Schwimmer E, Viswanathan M, Lohr K N. Defining treatment-resistant depression. Depress Anxiety. 2020; 37(2):134-45. Epub 2019/10/23. doi: PubMed PMID: 31638723.
[6] George M S, Lisanby S H, Avery D, McDonald W M, Durkalski V, Pavlicova M, Anderson B, Nahas Z, Bulow P, Zarkowski P, Holtzheimer P E, 3rd, Schwartz T, Sackeim H A. Daily left prefrontal transcranial magnetic stimulation therapy for major depressive disorder: a sham-controlled randomized trial. Arch Gen Psychiatry. 2010; 67(5):507-16. Epub 2010/05/05. doi: 67/5/507 [pii]10.1001/archgenpsychiatry.2010.46. PubMed PMID: 20439832.
[7] O'Reardon J P, Solvason H B, Janicak P G, Sampson S, Isenberg K E, Nahas Z, McDonald W M, Avery D, Fitzgerald P B, Loo C, Demitrack M A, George M S, Sackeim H A. Efficacy and safety of transcranial magnetic stimulation in the acute treatment of major depression: a multisite randomized controlled trial. Biol Psychiatry. 2007; 62(11):1208-16. Epub 2007/06/19. doi: S0006-3223(07)00146-1 [pii]10.1016/j.biopsych.2007.01.018. PubMed PMID: 17573044.
[8] Gaynes B N, Lloyd S W, Lux L, Gartlehner G, Hansen R A, Brode S, Jonas D E, Swinson Evans T, Viswanathan M, Lohr K N. Repetitive transcranial magnetic stimulation for treatment-resistant depression: a systematic review and meta-analysis. J Clin Psychiatry. 2014; 75(5):477-89. Epub 2014/06/13. doi: 10.4088/JCP.13r08815. PubMed PMID: 24922485.
[9] Carpenter L L, Janicak P G, Aaronson S T, Boyadjis T, Brock D G, Cook I A, Dunner D L, Lanocha K, Solvason H B, Demitrack M A. Transcranial magnetic stimulation (TMS) for major depression: a multisite, naturalistic, observational study of acute treatment outcomes in clinical practice. Depress Anxiety. 2012; 29(7):587-96. Epub 2012/06/13. doi: 10.1002/da.21969. PubMed PMID: 22689344.
[10] Kozel F A, Hernandez M, Van Trees K, Phillips S, Hashimie J, Weisman M, Obregon D, Sanchez D L, Catalano G, Schultz S K. Clinical repetitive transcranial magnetic stimulation for veterans with major depressive disorder. Ann Clin Psychiatry. 2017; 29(4):242-8. Epub 2017/10/27. PubMed PMID: 29069109.
[11] Hernandez M J, Reljic T, Van Trees K, Phillips S, Hashimie J, Bajor L, Yehl J, McKenzie B C, Burke C, Sullivan G A, Kumar A, Sanchez D L, Catalano G, Kozel F A. Impact of Comorbid PTSD on Outcome of Repetitive Transcranial Magnetic Stimulation (TMS) for Veterans With Depression. J Clin Psychiatry. 2020; 81(4). Epub 2020/07/14. doi: PubMed PMID: 32659874.
[12] Madore M R, Kozel F A, Williams L M, Green L C, George M S, Holtzheimer P E, Yesavage J A, Philip N S. Prefrontal transcranial magnetic stimulation for depression in US military veterans—A naturalistic cohort study in the veterans health administration. J Affect Disord. 2022; 297:671-8. Epub 2021/10/24. doi: 10.1016/j.jad.2021.10.025. PubMed PMID: 34687780; PMCID: PMC8667345.
[13] Kozel F A, Nahas Z, DeBrux C, Molloy M, Lorberbaum J P, Bohning D E, Risch S C, George M S. How the distance from coil to cortex relates to age, motor threshold and possibly the antidepressant response to repetitive transcranial magnetic stimulation. J Neuropsychiatry Clin Neurosci. 2000; 12:376-84.
[14] McConnell K A, Nahas Z, Shastri A, Lorberbaum J P, Kozel F A, Bohning D E, George M S. The transcranial magnetic stimulation motor threshold depends on the distance from coil to underlying cortex: a replication in healthy adults comparing two methods of assessing the distance to cortex. Biological psychiatry. 2001; 49(5):454-9. Epub 2001/03/29. PubMed PMID: 11274657.
[15] Stokes M G, Chambers C D, Gould I C, Henderson T R, Janko N E, Allen N B, Mattingley J B. Simple metric for scaling motor threshold based on scalp-cortex distance: application to studies using transcranial magnetic stimulation. J Neurophysiol. 2005; 94(6):4520-7. Epub 2005/09/02. doi: 10.1152/jn.00067.2005. PubMed PMID: 16135552.
[16] Cukic M, Kalauzi A, Ilic T, Miskovic M, Ljubisavljevic M. The influence of coil-skull distance on transcranial magnetic stimulation motor-evoked responses. Exp Brain Res. 2009; 192(1):53-60. Epub 2008/09/13. doi: 10.1007/s00221-008-1552-0 [doi]. PubMed PMID: 18787813.
[17] Mosimann U P, Marre S C, Werlen S, Schmitt W, Hess C W, Fisch H U, Schlaepfer T E. Antidepressant Effects of Repetitive Transcranial Magnetic Stimulation in the Elderly: Correlation Between Effect Size and Coil-Cortex Distance. Archives of General Psychiatry. 2002; 59:560-1.
[18] Pallanti S, Cantisani A, Grassi G, Antonini S, Cecchelli C, Burian J, Cauli G, Quercioli L. rTMS age-dependent response in treatment-resistant depressed subjects: a mini-review. CNS Spectr. 2012; 17(1):24-30. Epub 2012/07/14. doi: 10.1017/S1092852912000417. PubMed PMID: 22790115.
[19] Caulfield K A, Li X, George M S. Four electric field modeling methods of Dosing Prefrontal Transcranial Magnetic Stimulation (TMS): Introducing APEX MT dosimetry. Brain Stimulation: Basic, Translational, and Clinical Research in Neuromodulation. 2021; 14(4):1032-4.
[20] Villringer A, Chance B. Non-invasive optical spectroscopy and imaging of human brain function. Trends Neurosci. 1997; 20(10):435-42. Epub 1997/11/05. doi: S0166-2236(97)01132-6 [pii]. PubMed PMID: 9347608.
[21] Boas D A, Dale A M, Franceschini M A. Diffuse optical imaging of brain activation: approaches to optimizing image sensitivity, resolution, and accuracy. Neuroimage. 2004; 23 Suppl 1:S275-88. Epub 2004/10/27. doi: 51053-8119(04)00376-3 [pii]10.1016/j.neuroimage.2004.07.011 [doi]. PubMed PMID: 15501097.
[22] Kim J G, Liu H. Variation of haemoglobin extinction coefficients can cause errors in the determination of haemoglobin concentration measured by near-infrared spectroscopy. Phys Med Biol. 2007; 52(20):6295-322. Epub 2007/10/09. doi: 50031-9155(07)49281-8 [pii]10.1088/0031-9155/52/20/014 [doi]. PubMed PMID: 17921586.
[23] Kozel F A, Tian F, Dhamne S, Croarkin P E, McClintock S M, Elliott A, Mapes K S, Husain M M, Liu H. Using simultaneous repetitive Transcranial Magnetic Stimulation/functional Near Infrared Spectroscopy (rTMS/fNIRS) to measure brain activation and connectivity. Neuroimage. 2009; 47(4):1177-84. Epub 2009/05/19. doi: S1053-8119(09)00492-3 [pii]10.1016/j.neuroimage.2009.05.016 [doi]. PubMed PMID: 19446635.
[24] Allen E A, Pasley B N, Duong T, Freeman R D. Transcranial magnetic stimulation elicits coupled neural and hemodynamic consequences. Science. 2007; 317(5846):1918-21. PubMed PMID: 17901333.
[25] Shinba T, Kariya N, Matsuda S, Matsuda H, Obara Y. Increase of frontal cerebral blood volume during transcranial magnetic stimulation in depression is related to treatment effectiveness: A pilot study with near-infrared spectroscopy. Psychiatry Clin Neurosci. 2018; 72(8):602-10. Epub 2018/05/19. doi: 10.1111/pcn.12680. PubMed PMID: 29774621.
[26] Curtin A, Tong S, Sun J, Wang J, Onaral B, Ayaz H. A systematic review of integrated functional near-infrared spectroscopy (fNIRS) and transcranial magnetic stimulation (TMS) studies. Frontiers in Neuroscience. 2019; 13:84.
[27] Struckmann W, Persson J, Weigl W, Gingnell M, Boden R. Modulation of the prefrontal blood oxygenation response to intermittent theta-burst stimulation in depression: a sham-controlled study with functional near-infrared spectroscopy. The World Journal of Biological Psychiatry. 2021; 22(4):247-56.
[28] Jiang S, Huang J, Yang H, Wagoner R, Kozel F A, Currier G, Jiang H. Neuroimaging of depression with diffuse optical tomography during repetitive transcranial magnetic stimulation. Sci Rep. 2021; 11(1):7328. Epub 2021/04/03. doi: 10.1038/s41598-021-86751-9. PubMed PMID: 33795763; PMCID: PMC8016845.
[29] Tian F, Kozel F A, Yennu A, Croarkin P E, McClintock S M, Mapes K S, Husain M M, Liu H. Test-retest assessment of cortical activation induced by repetitive transcranial magnetic stimulation with brain atlas-guided optical topography. Journal of biomedical optics. 2012; 17(11):116020. Epub 2012/11/10. doi: 10.1117/1.JBO.17.11.116020. PubMed PMID: 23139044.
[30] Montgomery S A, Asberg M. A new depression scale designed to be sensitive to change. Br J Psychiatry. 1979; 134:382-9. PubMed PMID: 444788.
[31] Posner K, Brown G K, Stanley B, Brent D A, Yershova K V, Oquendo M A, Currier G W, Melvin G A, Greenhill L, Shen S, Mann J J. The Columbia-Suicide Severity Rating Scale: initial validity and internal consistency findings from three multisite studies with adolescents and adults. Am J Psychiatry. 2011; 168(12):1266-77. Epub 2011/12/24. doi: PubMed PMID: 22193671; PMCID: PMC3893686.
[32] Kroenke K, Spitzer R L. The PHQ-9: A new depression diagnostic and severity measure. Psychiat Ann. 2002; 32(9):509-15. doi: Doi 10.3928/0048-5713-20020901-06. PubMed PMID: WOS:000178070800004.
[33] Kroenke K, Spitzer R L, Williams J B. The PHQ-9: validity of a brief depression severity measure. J Gen Intern Med. 2001; 16(9):606-13. Epub 2001/09/15. doi: 10.1046/j.1525-1497.2001.016009606.x. PubMed PMID: 11556941; PMCID: PMC1495268.
[34] Spitzer R L, Kroenke K, Williams J B, Lowe B. A brief measure for assessing generalized anxiety disorder: the GAD-7. Arch Intern Med. 2006; 166(10):1092-7. Epub 2006/05/24. doi: PubMed PMID: 16717171.
[35] Blevins C A, Weathers F W, Davis M T, Witte T K, Domino J L. The Posttraumatic Stress Disorder Checklist for DSM-5 (PCL-5): Development and Initial Psychometric Evaluation. J Trauma Stress. 2015; 28(6):489-98. Epub 2015/11/26. doi: 10.1002/jts.22059. PubMed PMID: 26606250.
[36] Wortmann J H, Jordan A H, Weathers F W, Resick P A, Dondanville K A, Hall-Clark B, Foa E B, Young-McCaughan S, Yarvis J S, Hembree E A, Mintz J, Peterson A L, Litz B T. Psychometric analysis of the PTSD Checklist-5 (PCL-5) among treatment-seeking military service members. Psychol Assess. 2016; 28(11):1392-403. Epub 2016/11/04. doi: PubMed PMID: 26751087.
[37] Ware J E, Jr., Sherbourne C D. The MOS 36-item short-form health survey (SF-36). I. Conceptual framework and item selection. Med Care. 1992; 30(6):473-83. Epub 1992/06/11. PubMed PMID: 1593914.
[38] Brazier J E, Harper R, Jones N M, O'Cathain A, Thomas K J, Usherwood T, Westlake L. Validating the SF-36 health survey questionnaire: new outcome measure for primary care. BMJ. 1992; 305(6846):160-4. Epub 1992/07/18. doi: 10.1136/bmj.305.6846.160. PubMed PMID: 1285753; PMCID: PMC1883187.
[39] Rizvi S J, Quilty L C, Sproule B A, Cyriac A, Michael Bagby R, Kennedy S H. Development and validation of the Dimensional Anhedonia Rating Scale (DARS) in a community sample and individuals with major depression. Psychiatry Res. 2015; 229(1-2):109-19. Epub 2015/08/08. doi: 10.1016/j.psychres.2015.07.062. PubMed PMID: 26250147.
[40] Drakaki M, Mathiesen C, Siebner H R, Madsen K, Thielscher A. Database of 25 validated coil models for electric field simulations for TMS. Brain Stimul. 2022; 15(3):697-706. Epub 2022/05/02. doi: 10.1016/j.brs.2022.04.017. PubMed PMID: 35490970.
[41] Konakanchi D, de Jongh Curry A L, Waters R S, Narayana S. Focality of the Induced E-Field Is a Contributing Factor in the Choice of TMS Parameters: Evidence from a 3D Computational Model of the Human Brain. Brain Sci. 2020; 10(12). Epub 2020/12/24. doi: PubMed PMID: 33353125; PMCID: PMC7766380.
[42] Kammer T, Beck S, Thielscher A, Laubis-Herrmann U, Topka H. Motor thresholds in humans: a transcranial magnetic stimulation study comparing different pulse waveforms, current directions and stimulator types. Clin Neurophysiol. 2001; 112(2):250-8. Epub 2001/02/13. doi: 10.1016/s1388-2457(00)00513-7. PubMed PMID: 11165526.
[43] Johnson K A, Baig M, Ramsey D, Lisanby S H, Avery D, McDonald W M, Li X, Bernhardt E R, Haynor D R, Holtzheimer P E, 3rd, Sackeim H A, George M S, Nahas Z. Prefrontal rTMS for treating depression: location and intensity results from the OPT-TMS multi-site clinical trial. Brain Stimul. 2013; 6(2):108-17. Epub 2012/04/03. doi: 10.1016/j.brs.2012.02.003. PubMed PMID: 22465743; PMCID: PMC3573221.
[44] Zimeo Morais G A, Balardin J B, Sato J R. fNIRS Optodes' Location Decider (fOLD): a toolbox for probe arrangement guided by brain regions-of-interest. Scientific Reports. 2018; 8(1):3341. doi: 10.1038/s41598-018-21716-z.
[45] Brigadoi S, Salvagnin D, Fischetti M, Cooper R. Array Designer: automated optimized array design for functional near-infrared spectroscopy. Neurophotonics. 2018; 5(3):035010.
[46] Machado A, Cai Z, Pellegrino G, Marcotte O, Vincent T, Lina J M, Kobayashi E, Grova C. Optimal positioning of optodes on the scalp for personalized functional near-infrared spectroscopy investigations. Journal of Neuroscience Methods. 2018; 309:91-108. doi: https://doi.org/10.1016/j.jneumeth.2018.08.006.
[47] Zhao Y, Xiao X, Jiang Y-H, Sun P-P, Zhang Z, Gong Y-L, Li Z, Zhu C-Z. Transcranial brain atlas-based optimization for functional near-infrared spectroscopy optode arrangement: Theory, algorithm, and application. Human Brain Mapping. 2021; 42(6):1657-69. doi: https://doi.org/10.1002/hbm.25318.
[48] Fu X, Richards J. devfOLD: a toolbox for designing age-specific fNIRS channel placement. Neurophotonics. 2021; 8(4):045003.
[49] Huppert T J, Diamond S G, Franceschini M A, Boas D A. HomER: a review of time-series analysis methods for near-infrared spectroscopy of the brain. Applied Optics. 2009; 48(10):D280-D98. doi: 10.1364/AO.48.00D280.
[50] Santosa H, Zhai X, Fishburn F, Huppert T. The NIRS Brain AnalyzlR Toolbox. Algorithms. 2018; 11(5):73. PubMed PMID: doi:10.3390/a11050073.
[51] Hocke L M, Oni I K, Duszynski C C, Corrigan A V, Frederick B D, Dunn J F. Automated Processing of fNIRS Data—A Visual Guide to the Pitfalls and Consequences. Algorithms. 2018; 11(5):67. PubMed PMID: doi:10.3390/a11050067.
[52] Huppert T J. Commentary on the statistical properties of noise and its implication on general linear models in functional near-infrared spectroscopy. Neurophotonics. 2016; 3(1):010401. doi: 10.1117/1.NPh.3.1.010401. PubMed PMID: 26989756; PMCID: PMC4773699.
[53] Kamran M A, Mannan M M N, Jeong M Y. Cortical Signal Analysis and Advances in Functional Near-Infrared Spectroscopy Signal: A Review. Frontiers in Human Neuroscience. 2016; 10. doi: 10.3389/fnhum.2016.00261.
[54] Pfeifer M D, Scholkmann F, Labruyere R. Signal Processing in Functional Near-Infrared Spectroscopy (fNIRS): Methodological Differences Lead to Different Statistical Results. Frontiers in Human Neuroscience. 2018; 11. doi: 10.3389/fnhum.2017.00641.
[55] Pinti P, Scholkmann F, Hamilton A, Burgess P, Tachtsidis I. Current Status and Issues Regarding Pre-processing of fNIRS Neuroimaging Data: An Investigation of Diverse Signal Filtering Methods Within a General Linear Model Framework. Frontiers in Human Neuroscience. 2019; 12. doi: 10.3389/fnhum.2018.00505.
[56] von Liihmann A, Li X, Müller K-R, Boas D A, Yücel M A. Improved physiological noise regression in fNIRS: A multimodal extension of the General Linear Model using temporally embedded Canonical Correlation Analysis. Neurolmage. 2020; 208:116472. doi: https://doi.org/10.1016/j.neuroimage.2019.116472.
[57] von Liihmann A, Ortega-Martinez A, Boas D A, Yücel M A. Using the General Linear Model to Improve Performance in fNIRS Single Trial Analysis and Classification: A Perspective. Frontiers in Human Neuroscience. 2020; 14. doi: 10.3389/fnhum.2020.00030.
[58] Pollonini L, Bortfeld H, Oghalai J S. PHOEBE: a method for real time mapping of optodes-scalp coupling in functional near-infrared spectroscopy. Biomedical Optics Express. 2016; 7(12):5104-19. doi: 10.1364/BOE.7.005104.
[59] Molavi B, Dumont G A. Wavelet-based motion artifact removal for functional near-infrared spectroscopy. Physiol Meas. 2012; 33(2):259-70. doi: 10.1088/0967-3334/33/2/259.)
[60] Yücel M A, Selb J, Cooper R J, Boas D A. Targeted principle component analysis: A new motion artifact correction approach for near-infrared spectroscopy. Journal of Innovative Optical Health Sciences. 2014; 07(02):1350066. doi: 10.1142/s1793545813500661.
[61] Delpy D T, Cope M, van der Zee P, Arridge S R, Wray S, Wyatt J S. Estimation of optical pathlength through tissue from direct time of flight measurement. Physics in medicine and biology. 1988; 33 12:1433-42.
[62] Scholkmann F, Wolf M. General equation for the differential pathlength factor of the frontal human head depending on wavelength and age. Journal of Biomedical Optics. 2013; 18(10):105004.
[63] Santosa H, Aarabi A, Perlman S B, Huppert T J. Characterization and correction of the false-discovery rates in resting state connectivity using functional near-infrared spectroscopy. J Biomed Opt. 2017; 22(5):55002. Epub 2017/05/12. doi: 10.1117/11B0.22.5.055002. PubMed PMID: 28492852; PMCID: PMC5424771.
[64] Wang J-L, Chiou J-M, Müller H-G. Functional Data Analysis. Annual Review of Statistics and Its Application. 2016; 3(1):257-95. doi: 10.1146/annurev-statistics-041715-033624.
[65] Barker J W, Aarabi A, Huppert T J. Autoregressive model based algorithm for correcting motion and serially correlated errors in fNIRS. Biomed Opt Express. 2013; 4(8):1366-79. Epub 2013/09/07. doi: 10.1364/BOE.4.001366. PubMed PMID: 24009999; PMCID: PMC3756568.
[66] Benjamini Y, Hochberg Y. Controlling the False Discovery Rate: A Practical and Powerful Approach to Multiple Testing. Journal of the Royal Statistical Society: Series B (Methodological). 1995; 57(1):289-300. doi: https://doi.org/10.1111/j.2517-6161.1995.tb02031.x.
[67] Brigadoi S, Cooper R. How short is short? Optimum source—detector distance for short-separation channels in functional near-infrared spectroscopy. Neurophotonics. 2015; 2(2):025005.
[68] Wyser D, Mattille M, Wolf M, Lambercy O, Scholkmann F, Gassert R. Short-channel regression in functional near-infrared spectroscopy is more effective when considering heterogeneous scalp hemodynamics. Neurophotonics. 2020; 7(3):035011.
[69] Cui X, Bray S, Reiss A L. Functional near infrared spectroscopy (NIRS) signal improvement based on negative correlation between oxygenated and deoxygenated hemoglobin dynamics. Neurolmage. 2010; 49(4):3039-46. doi: https://doi.org/10.1016/j.neuroimage.2009.11.050.
[70] Tak S, Ye J C. Statistical analysis of fNIRS data: A comprehensive review. Neurolmage. 2014; 85:72-91. doi: https://doi.org/10.1016/j.neuroimage.2013.06.016.
[71] Welvaert M, Rosseel Y. On the Definition of Signal-To-Noise Ratio and Contrast-To-Noise Ratio for fMRI Data. PLOS ONE. 2013; 8(11):e77089. doi: 10.1371/journal.pone.0077089.
[72] Cooper R, Selb J, Gagnon L, Phillip D, Schytz H, Iversen H, Ashina M, Boas D. A Systematic Comparison of Motion Artifact Correction Techniques for Functional Near-Infrared Spectroscopy. Frontiers in Neuroscience. 2012; 6. doi: 10.3389/fnins.2012.00147.
[73] Pomann G-M, Staicu A-M, Ghosh S. A two-sample distribution-free test for functional data with application to a diffusion tensor imaging study of multiple sclerosis. Journal of the Royal Statistical Society: Series C (Applied Statistics). 2016; 65(3):395-414. doi: https://doi.org/10.1111/rssc.12130.
[74] Melendez R, Giraldo R, Leiva V. Sign, Wilcoxon and Mann-Whitney Tests for Functional Data: An Approach Based on Random Projections. Mathematics. 2021; 9(1):44. PubMed PMID: doi:10.3390/math9010044.
[75] Smaga L. A note on repeated measures analysis for functional data. AStA Advances in Statistical Analysis. 2020; 104(1):117-39. doi: 10.1007/s10182-018-00348-8.
[76] Wang Q. Two-sample inference for sparse functional data. Electronic Journal of Statistics. 2021; 15(1):1395-423, 29.
[77] van den Heuvel M P, Hulshoff Pol H E. Exploring the brain network: A review on resting-state fMRI functional connectivity. European Neuropsychopharmacology. 2010; 20(8):519-34. doi: https://doi.org/10.1016/j.euroneuro.2010.03.008.
[78] Wang H E, Bénar CG, Quilichini P P, Friston K J, Jirsa V K, Bernard C. A systematic framework for functional connectivity measures. Frontiers in Neuroscience. 2014; 8. doi:
[79] Hullait H, Leslie D S, Pavlidis N G, King S. Robust Function-on-Function Regression. Technometrics. 2021; 63(3):396-409. doi: 10.1080/00401706.2020.1802350.
[80] Morris J S. Functional Regression. Annual Review of Statistics and Its Application. 2015; 2(1):321-59. doi: 10.1146/annurev-statistics-010814-020413.
[81] Ferraty F, Van Keilegom I, Vieu P. Regression when both response and predictor are functions. Journal of Multivariate Analysis. 2012; 109:10-28. doi: https://doi.org/10.1016/j.jmva.2012.02.008.
[82] Janssens S E W, de Graaf T A, Duecker F, Schuhmann T, Sack A T. Assessing TMS-evoked cognitive network responses depending on neurocognitive and oscillatory brain state: A simultaneous TMS-EEG-fMRI project. bioRxiv. 2022:2022.04.07.487517. doi: 10.1101/2022.04.07.487517.

What is claimed is:

1. A method comprising:
measuring, via a functional Near-Infrared Spectroscopy (fNIRS) device, a first set of measurements of a treatment region of a patient to be treated;
performing, via a transcranial magnetic stimulation (TMS) device, TMS treatment to the treatment region for therapeutic effect;
measuring, via the fNIRS device, a second set of measurements of the treatment region;
determining, by a processor, an applied dosing of the TMS treatment based on at least one of the first set of measurements or the second set of measurements; and
outputting control feedback to the TMS device or visualization to a display to adjust one or more parameters or localization of the TMS treatment based on the applied dosing.

2. The method of claim 1, wherein the adjustment includes a coil to treatment region distance adjustment.

3. The method of claim 1, wherein the adjustment includes a gyms orientation adjustment.

4. The method of claim 1, wherein the treatment region is the prefrontal cortex and the adjustment includes a cortical excitability output to a motor cortex and dorsolateral prefrontal cortex.

5. The method of claim 1, wherein the adjustment includes a location of the TMS treatment.

6. The method of claim 1, wherein the adjustment includes a frequency of the TMS treatment.

7. The method of claim 1, wherein the adjustment includes a duration of the TMS treatment.

8. The method of claim 1, wherein the control feedback to the TMS device or visualization to the display is used to monitor an effectiveness of treatment changes based on the TMS treatment.

9. The method of claim 8, wherein the effectiveness of treatment region changes includes monitoring changes to one or more of moderator or mediators.

10. The method of claim 1, wherein the control feedback to the TMS device or visualization to the display is used to determine a stopping criteria for the TMS treatment.

11. The method of claim 1, wherein the TMS device is configured to generate at least one of: a 12-block sequence of 1 Hz stimulation with 4 sec on and off 26 sec, a 12-block sequence of 10 Hz stimulation with 4 sec on and off 26 sec; or a 48-jittered single pulses stimulation sequence.

12. The method of claim 1, wherein the fNIRS device includes a plurality of light sources and a plurality of light detectors, at least one of each forming a pair, wherein each pair is configured to be placed a predetermined distance apart from one another.

13. The method of claim 1, wherein the TMS treatment is for treatment of depression, the treatment region is the prefrontal cortex, and wherein the control feedback to the TMS device or visualization to the display is to provide sufficient cortical excitability for depression treatment.

14. A system comprising:
a functional Near-Infrared Spectroscopy (fNIRS) device;
a transcranial magnetic stimulation (TMS) device; and
an integrated component having a processor and a memory having instructions stored thereon, wherein execution of the instructions by the processor causes the processor to:
receive a first set of fNIRS measurements of a treatment region in response to a TMS treatment applied by the TMS device to the treatment region of a patient;
receive a second set of fNIRS measurements of the treatment region;
determine an applied dosing of the TMS treatment based on one or more of the first set of fNIRS or the second set of fNIRS; and
provide control feedback to the TMS device or visualization to a display to adjust one or more parameters or localization of the TMS treatment based on the applied dosing.

15. The system of claim 14, wherein the instruction further causes the processor to provide control feedback to the TMS device or visualization to the display to adjust one or more of a coil to treatment region distance, a gyms orientation, an excitability output, a location of the TMS treatment, a frequency of the TMS treatment, a duration of the TMS treatment, an effectiveness of treatment region changes based on the TMS treatment, or a stopping criteria for the TMS treatment.

16. The system of claim 14, wherein the TMS device is configured to generate at least one of: a 12-block sequence of 1 Hz stimulation with 4 sec on and off 26 sec, a 12-block sequence of Hz stimulation with 4 sec on and off 26 sec; or a 48-jittered single pulses stimulation sequence.

17. The system of claim 14, wherein the fNIRS device includes a plurality of light sources and a plurality of light detectors, at least one of each forming a pair, wherein each pair is configured to be placed a predetermined distance apart from one another.

18. A non-transitory computer-readable medium for a transcranial magnetic stimulation (TMS) functional Near-Infrared Spectroscopy (fNIRS) (TMS-fNIRS), system the non-transitory computer-readable medium having instructions stored thereon, wherein execution of the instructions by one or more processors causes the one or more processors to:
receive a first set of fNIRS measurements from a fNIRS device of the TMS-fNIRS system of a treatment region in response to a TMS treatment applied by a TMS device of the TMS-fNIRS system to the treatment region of a patient;
receive a second set of fNIRS measurements from the fNIRS device of the treatment region;
determine an applied dosing of the TMS treatment based on one or more of the first set of fNIRS or the second set of fNIRS; and
provide control feedback to the TMS device or visualization to a display to adjust one or more parameters or localization of the TMS treatment based on the applied dosing.

19. The non-transitory computer-readable medium of claim 18, wherein the instruction further causes the processor to provide control feedback to the TMS device or visualization to the display to adjust one or more of a coil to treatment region distance, a gyms orientation, an excitability output, a location of the TMS treatment, a frequency of the TMS treatment, a duration of the TMS treatment, an effectiveness of treatment region changes based on the TMS treatment, or a stopping criteria for the TMS treatment.

20. The non-transitory computer-readable medium of claim 18, wherein the TMS treatment is for treatment of depression, the treatment region is the prefrontal cortex, and wherein the control feedback to the TMS system or visualization to the display is to provide sufficient cortical excitability for depression treatment.
